(19) United States
(12) Patent Application Publication
(10) Pub. No.: US 2012/0035219 A1
(43) Pub. Date: Feb. 9, 2012

(54) Compound for the Treatment of Tuberculosis

(75) Inventors: Kaveri Das, Bangalore (IN); David Alan Melnick, Wilmington, DE (US); Shandil Radha, Bangalore (IN)

(73) Assignee: ASTRAZENECA AB, Södertälje (SE)

(21) Appl. No.: 13/256,658

(22) PCT Filed: Mar. 16, 2010
(86) PCT No.: PCT/GB2010/050445

§ 371 Date: Oct. 25, 2011

Related U.S. Application Data
(60) Provisional application No. 61/160,385, filed on Mar. 16, 2009.

(51) Int. Cl. A61K 31/4439; A61P 31/06; A01P 1/00; C07D 413/14; A01N 43/80

(52) U.S. Cl. 514/340; 546/271.4

(57) ABSTRACT

(5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one, or a pharmaceutically-acceptable salt, or an in-vivo-hydrolysable ester thereof, for use in the treatment of Mycobacterium tuberculosis.

The present invention relates to the use of AZD2563 in the treatment of tuberculosis.

Tuberculosis is a disease caused by Mycobacterium tuberculosis (Mtu), which in 1990 was declared a global epidemic by the World Health Organisation (WHO). It affects more than one third of the world's population resulting in 8 million new patients and 2 million deaths every year. Also there exists a scenario called “Latent TB”, which occurs when germs remain in the body in a quiescent state but without any apparent effect on the health of the individual. In many cases this stage may last for many years or decades. In case of normal human being the chance of activation is 2-23% in a lifetime. However in case of immuno-compromised patients (like HIV) the chances of activation rise to 10% every year.

The current treatment of drug sensitive tuberculosis is at least six months long and requires a combination of isoniazid, rifampicin, pyrazinamide and ethambutol in the first two months followed by isoniazid and rifampicin for a period of four months. In recent years, drug resistance to these drugs has increased and the last of drugs for tuberculosis was introduced into clinical practice in the late 1960's. The evolution of resistance could result in strains against which currently available antitubercular agents will be ineffective and treatment in such cases may last two years with no guarantee of cure. So there is an urgent need to introduce new drugs particularly those with either a novel mechanism of action and/or containing new pharmacophoric groups and new treatment regimens to overcome not only rising drug resistance but also improve the overall treatment duration.

R. Sood et al (Infectious Disorders—Drug Targets 2006, 343-354) report that “Oxazolidinones are a new class of totally synthetic antibacterial agents with wide spectrum of activity against a variety of clinically significant susceptible and resistant bacteria. These compounds have been shown to inhibit translation at the initiation phase of protein synthesis. DuP-721, the first oxazolidinone showed good activity against M. tuberculosis when given orally or parenterally to experimental animals but was not developed further due to lethal toxicity in animal models. Later two oxazolidinones, PNU-100480 and Linezolid, demonstrated promising antimycobacterial activities in the murine model. While Linezolid has been approved for clinical use for broad spectrum area, PNU-100840 was not developed further. DA-7867 showed good in vitro and better in vivo efficacy than Linezolid but was poorly tolerated in rat toxicology studies. The antimycobacterial activity of AZD2563 has not been explored. RBx 7644 had modest antimycobacterial activity whilst RBx 8700 has potent antibacterial and concentration dependent activity against all slow growing mycobacteria. It demonstrated better activity than RBx 7644 against MDR strains of M. tuberculosis along with intracellular activity”.

In our published patent application WO-99/64417 we disclose the compound

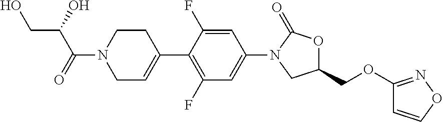

ie. (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one also known as AZD2563. As reported by R. Sood et al (op cit) the antimycobacterial activity of AZD2563 has not been explored.

In a first aspect of the invention we now provide (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one or a pharmaceutically-acceptable salt, or an in-vivo-hydrolysable ester thereof, for use in the treatment of Mycobacterium tuberculosis.

The compound can form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate. fumarate, hydrochloride, citrate, maleate, tartrate and hydrobromide. Also suitable are salts formed with phosphoric and sulfuric acid. In another aspect suitable salts are base salts such as an alkali metal salt for example sodium, an alkaline earth metal salt for example calcium or magnesium, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions. In one aspect of the invention the pharmaceutically-acceptable salt is the sodium salt.

However, to facilitate isolation of the salt during preparation, salts which are less soluble in the chosen solvent may be utilised whether pharmaceutically acceptable or not.

Within the present invention it is to be understood that the compound or a salt thereof may exhibit the phenomenon of tautomerism and that the formulae drawings within this specification can represent only one of the possible tautomeric forms. It is to be understood that the invention encompasses any tautomeric form and is not to be limited merely to any one tautomeric form utilised within the formulae drawings. The formulae drawings within this specification can represent only one of the possible tautomeric forms and it is to be understood that the specification encompasses all possible tautomeric forms of the compounds drawn not just those forms which it has been possible to show graphically herein. The same applies to compound names.

It is to be understood that the present invention encompasses any racemic, optically-active, polymorphic or stereoisomeric form, or mixtures thereof, at any additional asymmetrically substituted carbon(s) and sulphur atom(s), which possesses anti-tubercular properties

Optically-active forms may be prepared by procedures known in the art for example, by resolution of the racemic form by re-crystallisation techniques, by synthesis from optically-active starting materials, by chiral synthesis, by enzymatic resolution, by biotransformation, or by chromatographic separation using a chiral stationary phase.

The compound may exhibit polymorphism. It is to be understood that the present invention encompasses any polymorphic form, or mixtures thereof, which form possesses which possesses anti-tubercular properties

It is also to be understood that the compound of the invention and salts thereof can exist in solvated as well as unsolvated forms such as, for example, hydrated forms. It is to be understood that the invention encompasses the use of all such solvated forms, which possesses anti-tubercular properties.

Our investigations have shown AZD2563 can act as an anti-tubercular agent for a longer time with lower exposure when compared with linezolid. This may allow once daily dosing, whereas linezolid is dosed twice a day. Whilst we do not wish to be bound by theoretical considerations this may provide an improved safety profile.

In a further aspect of the invention we provide the use of AZD2563 or a pharmaceutically-acceptable salt, or an in-vivo-hydrolysable ester thereof in the preparation of a medicament for use in the treatment of Mycobacterium tuberculosis.

In a further aspect of the invention we provide a method for the attenuation of Mycobacterium tuberculosis which method comprises contacting cells infected by Mycobacterium tuberculosis with a pharmaceutically effective amount of AZD2563 or a pharmaceutically-acceptable salt, or an in-vivo-hydrolysable ester thereof whereby the cells are attenuated.

In a further aspect of the invention we provide a method for the treatment of Mycobacterium tuberculosis which method comprises administering a therapeutically effective amount of AZD2563 or a pharmaceutically-acceptable salt, or an in-vivo-hydrolysable ester thereof to a patient in need of anti-tubercular therapy.

In a further aspect of the invention (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one or a pharmaceutically-acceptable salt, or an in-vivo-hydrolysable ester thereof, is administered no more than once daily.

In a further aspect of the invention we provide a pharmaceutical formulation comprising (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one or a pharmaceutically-acceptable salt, or an in-vivo-hydrolysable ester thereof, for administration no more than once daily.

It will be understood that AZD2563 may be used for both human and animal therapy. Each represents an independent aspect of the invention.

### Combinations

The compound of the invention described herein may be applied as a sole therapy or may involve, in addition to a compound of the invention, one or more other substances and/or treatments. Such conjoint treatment may be achieved by way of the simultaneous, sequential or separate administration of the individual components of the treatment. Where the administration is sequential or separate, the delay in administering the second component should not be such as to lose the beneficial effect of the combination.

Suitable classes and substances may be selected from one or more of the following:

i) other antibacterial agents for example macrolides e.g. erythromycin, azithromycin capreomycin or clarithromycin; quinolones e.g. ciprofloxacin or levofloxacin; β-lactams e.g. penicillins e.g. amoxicillin or piperacillin; cephalosporins e.g. ceftriaxone or ceftazidime; carbapenems, e.g. meropenem or imipenem etc; aminoglycosides e.g. gentamicin or tobramycin; or oxazolidinones; and/or
ii) anti-infective agents for example, an antifungal triazole e.g. or amphotericin; and/or
iii) biological protein therapeutics for example antibodies, cytokines, bactericidal/permeability-increasing protein (BPI) products;
and/or
iv) one or more antibacterial agents useful in the treatment of tuberculosis such as one or more of rifampicin, isoniazid, pyrazinamide, ethambutol, quinolones e.g. moxifloxacin or gatifloxacin, streptomycin, cycloserine, ethionamide, thiacetazone, p-aminosalicylic acid (PAS), amikacin, kanamycin, clofazimine
v) efflux pump inhibitors.

Therefore in a further aspect of the invention we provide a combination therapy comprising (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one, or a pharmaceutically acceptable salt thereof in combination with a chemotherapeutic agent selected from:

i) one or more additional antibacterial agents; and/or ii) one or more anti-infective agents; and/or iii) biological protein therapeutics for example antibodies, cytokines, bactericidal/permeability-increasing protein (BPI) products; iv) one or more antibacterial agents useful in the treatment of tuberculosis and/or v) one or more efflux pump inhibitors.

In a further aspect of the invention the combination therapy is provided for administration no more than once daily.

The invention will now be illustrated but not limited to the following specific description

Synthesis of 5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one (AZD 2563) is disclosed in our published patent application WO-99/64417.

As a comparator we profiled linezolid (marketed as Zyvox®) which is N—[(S)-3-(3-Fluoro-4-morpholin-4-yl-phenyl)-2-oxo-oxazolidin-5-ylmethyl]-acetamide

### BIOLOGICAL TESTING PROCEDURES

### Bacterial Susceptibility Testing Methods

The compound may be tested for antimicrobial activity by susceptibility testing in liquid media. Compounds may be dissolved in dimethylsulfoxide and tested in 10 doubling dilutions in the susceptibility assays. The organisms used in the assay may be grown overnight on suitable agar media and then suspended in a liquid medium appropriate for the growth of the organism. The suspension can be a 0.5 McFarland and a further 1 in 10 dilution can be made into the same liquid medium to prepare the final organism suspension in 100 μL. Plates can be incubated under appropriate conditions at 37° C. for 24 hrs prior to reading. The Minimum Inhibitory Concentration (MIC) may be determined as the lowest drug concentration able to reduce growth by 90% or more.

### In Vitro Mycobacteria Susceptibility Testing Methods

Protocol for MIC testing: Microplate Alamar Blue Assay (Franzblau et al, 1998. J. Clin. Microbiol. 36: 362-366). Bacterial Culture: Mtu H37Rv (ATCC 27294) was stored at −70 C and used for infection.

Two hundred microliters of sterile deionised water was added to all outer-perimeter wells of sterile 96-well plates to minimize evaporation of the medium in the test wells during incubation. Serial two-fold dilutions of the compounds in DMSO were made in another 96 well plate starting from 64 μg/ml to 0.5 μg/ml. 4 ul volumes of these were dispensed into the test wells in rows B to G in columns 2 to 10 by using a multichannel pipette. 200 μL of Mtu culture diluted to a cell number of about 5×10⁵ cfu/ml was added to all the wells and the contents of the wells were mixed well. Three wells in column 11 served as drug-free (inoculum-only) controls. And 3 wells served as drug-free medium controls. The plates were incubated at 37 deg C. for 5 days. Fifty microliters of a freshly prepared 1:1 mixture of Alamar Blue (Accumed International, Westlake, Ohio) reagent and 10% Tween 80 was added to well B11. The plates were reincubated at 37° C. for 24 h. If well B11 turned pink, the reagent mixture was added to all wells in the microplate (if the well remained blue, the reagent mixture would be added to another control well and the result would be read on the following day). The microplates were re-incubated for an additional 24 h at 37° C., and the colours of all wells were recorded. A blue colour in the well was interpreted as no growth, and a pink colour was scored as growth.

The MIC was defined as the lowest drug concentration, which prevented a colour change from blue to pink.

Once results from the above experiment are obtained, repeat MIC assays in duplicate is set up for determination of minimum bactericidal concentration (MBC). After incubation as detailed above, 100 ul from corresponding MIC well from the second plate, as well from wells, up to the highest concentration, is plated on 7H10 agar Plates. These plates are incubated at 37° C. for 15-20 days. Aliquot from positive control wells serve as growth control and 100 ul aliquot from media wells serve as negative control. After 21 days, each plate is visually or with a colony counter scored for colony forming units (CFU). The concentration at which less then 50 colonies are obtained is called MBC.

### Bone Marrow Derived Macrophages (BMDM) Culture and Infection

Bone marrow derived macrophages were obtained from BALB/c mice. Mice were euthanized by exposure to CO₂ and the femur and tibia were dissected out. The bones were trimmed at each end and the marrow was flushed out with cold RPMI 1640 medium using 26-gauge needle. Cell suspensions were then washed twice with medium and plated at 2×10⁶ cells/ml in 24 well plates in RPMI 1640 medium supplemented with 10% fetal bovine serum (FBS) and 20% culture supernatant of L929 (mouse fibroblast cell line). The cells were incubated at 37° C. in 5% CO₂ for 7 days with twice medium change.

Macrophages were used for infection with Mtu on 8^{th} day of culture. They were infected with MOI of 1:10 (macrophage:bacteria) for 2 hours in the regular culture conditions. After 2 hours the monolayers were thoroughly washed with pre-warmed Phosphate buffered Saline twice, to remove any extra cellular bacteria and replaced with RPMI 1640 with all the supplements containing drugs of different concentrations. (0.5 to 8 mg/L). The plates containing cells were periodically observed to note any changes in the cell morphology (due to drug toxicity) or lysis of monolayer. After 10 days of drug exposure (post infection) the monolayers were gently washed and lysed with 0.04% SDS and plated onto nutrient 7H11 agar. Bacterial colony formation was enumerated after incubation of plates for 21 days at 37° C., 5% CO₂ in humidified atmosphere. Data were expressed as the mean Log₁₀mean CFU for each drug concentration and the entire experiment was repeated twice.

### In Vivo Dose-Response Studies (Efficacy Testing Methods)

An aerosol infection model in which the effects of drugs are evaluated following a respiratory infection with small numbers of tubercle bacilli was used. Mice were infected in a biosafety level 3 facility via the inhalation route in an aerosol infection chamber. BALB/c mice (8-10 week old) were infected via respiratory route to achieve 100-200 bacilli/animal on the day of infection. At 4 weeks post infection, the mice were dosed once per day orally 100-125 mg/kg of body weight, with therapy given 6 days a week for 4 weeks. At the onset and 24 h after the completion of treatment, groups of mice were killed by exposure to CO₂, and the lungs were aseptically removed for homogenization in a final volume of 3 ml by using Wheaton Teflon-Glass tissue grinders (catalogue no. W012576). Each suspension was serially diluted in 10-fold steps, and at least 3 dilutions were plated onto Middlebrook 7H11 agar supplemented with 10% albumin dextrose catalase (Difco Laboratories) and incubated at 37° C. with 5% CO₂ for 3 weeks. The plates are enumerated for colony forming units (CFU) and the effect is compared against no treatment control.

### Statistical Analysis

The colony counts obtained from plating were transformed to Log₁₀(x+1), where x equals the total number of viable tubercle bacilli present in a given sample. Prism software (version 3; GraphPad Software, Inc., San Diego, Calif.) was used for statistical evaluations.

### Estimation of Pharmacokinetic (PK) Parameters

BALB/c mice (8-10 week old) were dosed with the compound in an appropriate vehicle at specified doses as a single dose at a dose volume of 10 mL/Kg. The dose was administered either orally or parentally. Blood samples were collected at various time points ranging from 0.08 h to 50 h post dosing and plasma harvested by acceptable methods like precipitation or extraction. The concentration of the compound in plasma was determined by standard analytical instruments like HPLC and/or LC-MS.

### PK Analysis:

PK analyses of the plasma concentration-time relationships were performed with WinNonLin software (or any other suitable software program). A noncompartmental analysis program was used to calculate the PK parameters, such as the maximum concentration of drug in plasma (Cₘₐₓ), time to Cₘₐₓ(tₘₐₓ), elimination rate constant, elimination half-life, and AUC from time zero to infinity (AUC_{0-inf})

AZD2563 was tested in the above-mentioned assays and the following results obtained:

TABLE 1
Comparison of Microbiological Activities for AZD2563 and Linezolid.

| | AZD2563 | Linezolid |
|---|---|---|
| Structure | 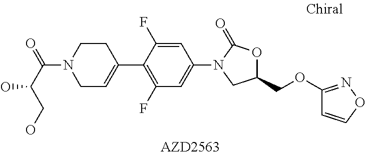 | 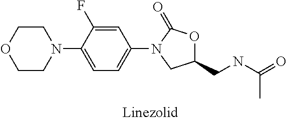 |
| MIC Mtu μg/mL | 0.5 | 0.5 |
| MBC Mtu μg/mL | 2.0 | 4 |
| BMDM (log Redn @ | 1.3 | −0.05 |
| 8 μg/mL) | | |
| Minimum Effective | 125 | 250 |
| Dose (mg/kg) in a | | |
| 4 wk efficacy model | | |
| AUC (hr. μg/mL) | 150.39 | 278.7 |
| PPB (Fraction free) | 0.18 | 0.70 |
| fAUC | 27.07 | 195.12 |
| fAUC/MIC | 54.14 | 390.24 |

Our investigations have shown, that AZD 2563 has greater bactericidal activity in the intracellular infection model (BMDM) as compared to Linezolid. Comparing the PK/PD indices in efficacy experiments, we see that, at the minimum effective dose, AZD2563 gives much lower exposure and requires lower fAUC/MIC but still yields similar potency when compared to Linezolid.

TABLE 2
Comparison of PK Properties for AZD2563
and Linezolid in various Species.

| | PK | | |
|---|---|---|---|
| Species | Parameter | Linezolid | AZD2563 |
| Mouse (10 mg/kg) | t_{1/2} | 1 | 1.3 |
| | PPB | 0.70 | 0.18 |
| | AUC | 7.3 | 20.3 |
| Rat | t_{1/2} | 1.3-3.0 | 4.0-6.0 |
| (20-25 mg/kg) | PPB | 0.78 | 0.13 |
| | AUC | 41.6 | 9 |
| Dog | t_{1/2} | 4 | 10 |
| (12.5-15 mg/kg) | PPB | 0.70 | 0.35 |
| | AUC | 42 | 42 |

The compounds were dosed to animals at various doses (not exceeding 25 mg/kg) and the concentration of the compound at various time points was estimated. The data was analysed and PK parameters like AUC. t_{1/2} and fAUC/MIC were estimated and the data shown in the table above. From the data the PK parameters such as longer t½ and lower exposure seem to hold in all the species and hence similar conclusions regarding toxicity and dosing intervals can be made in various species.

1. (canceled)

2. (canceled)

3. A method for the attenuation of Mycobacterium tuberculosis which method comprises contacting tubercular cells with a pharmaceutically effective amount of (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one whereby the cells are attenuated.

4. A method for the treatment of Mycobacterium tuberculosis which method comprises administering a therapeutically effective amount of (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one or a pharmaceutically-acceptable salt, or an in-vivo-hydrolysable ester thereof to a patient in need of anti-tubercular therapy.

5. The method as claimed in claim 4 and wherein (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one or a pharmaceutically-acceptable salt, or an in-vivo-hydrolysable ester thereof, is administered no more than once daily.

6. A pharmaceutical formulation comprising (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one or a pharmaceutically-acceptable salt, or an in-vivo-hydrolysable ester thereof, for administration no more than once daily.

7. A combination therapy comprising (5R)-3-[4-[1-[(2S)-2,3-dihydroxypropanoyl]-3,6-dihydro-2H-pyridin-4-yl]-3,5-difluoro-phenyl]-5-(isoxazol-3-yloxymethyl)oxazolidin-2-one, or a pharmaceutically acceptable salt thereof in combination with a chemotherapeutic agent selected from: i) one or more additional antibacterial agents; and/or ii) one or more anti infective agents; and/or iii) biological protein therapeutics; iv) one or more antibacterial agents useful in the treatment of tuberculosis and/or v) one or more efflux pump inhibitors.

8. The combination therapy as claimed in claim 7 for administration no more than once daily.

9. The combination therapy as claimed in claim 7 wherein the biological protein therapeutics are selected from antibodies, cytokines and bactericidal/permeability increasing protein (BPI) products.
